(19) United States
(12) Patent Application Publication
(10) Pub. No.: US 2025/0154447 A1
(43) Pub. Date: May 15, 2025

(54) Measurement System and Method for in Situ Calibration

(71) Applicant: Imec vzw, Leuven (BE)

(72) Inventor: Olivier Henry, Kraainem (BE)

(21) Appl. No.: 18/942,722

(22) Filed: Nov. 10, 2024

(30) Foreign Application Priority Data
Nov. 10, 2023 (EP) 23208978.9

(51) Int. Cl. C12M 1/34; C12M 1/00

(52) U.S. Cl. CPC C12M 41/00; C12M 29/04

(57) ABSTRACT

A measurement system is provided for in situ calibration. The measurement system includes a housing including a culture medium and measurement probe arranged in the culture medium. The measurement probe includes an opening, a sensor, and a channel arranged from the opening to the sensor. The measurement system further includes a calibration unit connected to the opening and configured to supply a calibration fluid to the sensor via the channel.

### CROSS-REFERENCE TO RELATED APPLICATIONS

The present application is a non-provisional patent application claiming priority to European patent application no. 23208978.9, filed on Nov. 10, 2023, the contents of which are hereby incorporated by reference.

### FIELD OF THE DISCLOSURE

The disclosure relates to in situ calibration of sensors, particularly of Process Analytical Technology (PAT) sensor probes for bioprocess monitoring.

### BACKGROUND

Generally, in drug bio-manufacturing, biological cells may be cultured under very controlled sterile conditions inside a bioreactor to maintain an optimal growth environment, where chemical and biochemical parameters may be monitored continuously, in situ. This can be achieved by using so-called “sensor probes” that may be placed inside the bioreactor. Furthermore, the sensors may be required to remain operational over the entire cell culture period, which may last somewhere between 3 and 6 weeks depending on the cell types.

However, current sensor technology generally cannot be operated for such an extended period without periodic calibration to guarantee the accuracy of the measurements. In addition, sensors cannot be taken out of the bioreactors to be calibrated, and re-inserted given the high risk of breaking sterility, i.e. introducing contamination into the cell culture.

In most settings, small samples may be collected from the reactor and may be analyzed off-line, and the results may be used to correct the in situ sensor readings. For example, the document U.S. Pat. No. 8,117,924 B2 discloses an apparatus that may isolate a sample from a bulk fluid to measure its characteristics. However, this may require skilled personnel, and access to additional analytical facilities that may not necessarily be available immediately.

### SUMMARY

Accordingly, a potential benefit of the disclosure is to provide a measurement system and a method to facilitate in situ calibration of measurement sensors without needing to collect samples.

According to a first aspect of the disclosure, a measurement system is provided for in situ calibration. The measurement system comprises a housing comprising a culture medium and at least one measurement probe arranged in the culture medium, wherein the at least one measurement probe comprises an opening, at least one sensor, and at least one channel arranged from the opening to the at least one sensor.

The measurement system further comprises a calibration unit connected to the opening, configured to supply a calibration fluid to the at least one sensor via the at least one channel. For instance, the calibration fluid may be sterilized before use. By means of the channel arranged in the measurement probe, the calibration unit may supply the calibration fluid to the sensor to perform in situ calibration without needing to collect samples of the culture medium.

For example, the at least one measurement probe comprises a membrane encompassing the at least one sensor, configured to confine the calibration fluid at or around the at least one sensor. A rapid diffusion and dilution of the calibration fluid into the culture medium may be prevented. Furthermore, the calibration accuracy may be improved, e.g., by confining the calibration fluid above the sensor during the calibration measurement.

For example, the calibration unit comprises a controller configured to control a flow of the calibration fluid in the at least one channel. In this regard, the flow can be bidirectional, i.e., from the calibration unit to the sensor as well as from the sensor to the calibration unit, especially within the channel.

For example, the controller is further configured to control a flow of the culture medium to the at least one sensor through the membrane. In this regard, the flow can be bidirectional, i.e., from the housing to the sensor as well as from the sensor to the housing, especially through the membrane.

For example, the membrane may protect the sensors from the culture medium. Furthermore, by controlling the flow of the culture medium through the membrane, the effect of flow during the measurement and/or calibration can be prevented or limited.

For example, the calibration fluid comprises a calibration solution with a defined concentration. For example, the calibration fluid may comprise a calibration solution for a specific sensor, and the calibration unit may comprise such calibration fluids for respective sensors.

For example, the calibration fluid comprises a plurality of calibration solutions with different concentrations. For example, the calibration fluid may comprise a combination of calibration solutions for calibrating multiple sensors, and the calibration unit may comprise such combined calibration solutions of different concentrations.

For example, the membrane comprises a plurality of pores with an internal width from 1 micrometer to 5 micrometers. For example, the membrane may comprise a plurality of pores with an internal width from 10 nm to 100 nm. For example, the pores may allow sample diffusion, e.g., proteins, at the sensor in a controlled manner, while preventing any contamination at the sensor surface.

For example, the calibration unit is further configured to supply a cleaning fluid to the at least one sensor via the at least one channel. For example, measurement and/or calibration accuracy can be further improved.

For example, the at least one measurement probe, the calibration fluid, and the cleaning fluid are sterile. For example, the sterile conditions of the bioprocess monitoring can be maintained.

For example, the at least one measurement probe further comprises at least one further sensor configured to be in contact with the culture medium. For example, by means of the further sensor in direct contact with the culture medium, i.e. external or not confined by the membrane, a differential measurement can be performed, which may further improve the measurement and/or calibration accuracy.

For example, the at least one sensor and/or the at least one further sensor are electrochemical sensors. Alternatively, the at least one sensor and/or the at least one further sensor are optical sensors. Further alternatively, the at least one sensor and/or the at least one further sensor are mass-sensitive sensors. Further alternatively, the at least one sensor and/or the at least one further sensor are thermo-sensitive sensors.

Additionally or alternatively, the at least one sensor and/or the at least one further sensor are configured to measure at least one of a physical parameter, a chemical parameter, or a biochemical parameter of the culture medium. The parameters can be defined as but not limited to temperature, pH level, dissolved oxygen, glucose, lactate, glutamine and glutamate concentrations, ion concentration (e.g. sodium, calcium, magnesium) cell density, cell viability, electrical conductivity, oxidation reduction potential, and proteins concentrations.

For example, the housing is a cell culture vessel, for instance a bioreactor vessel. Alternatively, the housing may correspond to a sterile environment for environmental and/or industrial monitoring.

According to a second aspect of the disclosure, a method is provided for in situ calibration. The method comprises the steps of providing a housing comprising a culture medium; arranging at least one measurement probe comprising an opening, at least one sensor, and at least one channel arranged from the opening to the at least one sensor in the culture medium; connecting a calibration unit to the opening; and supplying, by the calibration unit, a calibration fluid to the at least one sensor via the at least one channel.

For example, the method further comprises the step of providing a membrane encompassing the at least one sensor to confine the calibration fluid at or around the at least one sensor.

For example, the calibration unit comprises a controller, and the method further comprises the step of controlling, via the controller, a flow of the calibration fluid in the at least one channel. Additionally or alternatively, the method further comprises the step of controlling, via the controller, a flow of the culture medium to the at least one sensor through the membrane.

It is to be noted that the method according to the second aspect corresponds to the measurement system according to the first aspect and its implementation forms. Accordingly, the method of the second aspect may have corresponding implementation forms. Further, the method of the second aspect generally includes the same advantages and effects as the measurement system of the first aspect and its respective implementation forms.

### BRIEF DESCRIPTION OF THE FIGURES

The above, as well as additional, features will be better understood through the following illustrative and non-limiting detailed description of example embodiments, with reference to the appended drawings.

FIG. 1 shows a measurement system, according to an example;

FIG. 2 shows a calibration unit, according to an example;

FIG. 3 shows an in situ calibration operation, according to an example;

FIG. 4 shows a measurement system according to an example; and

FIG. 5 shows a method according to an example.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

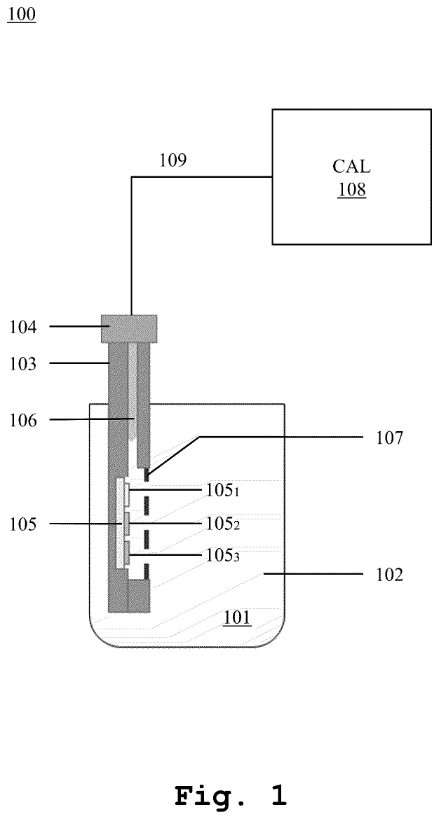

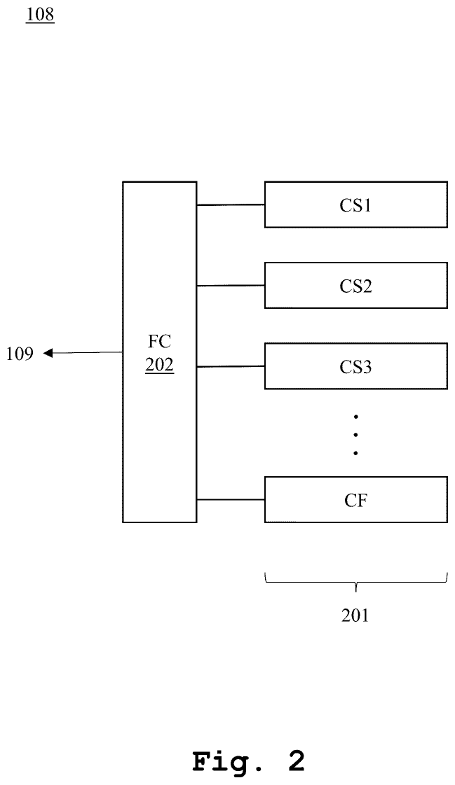

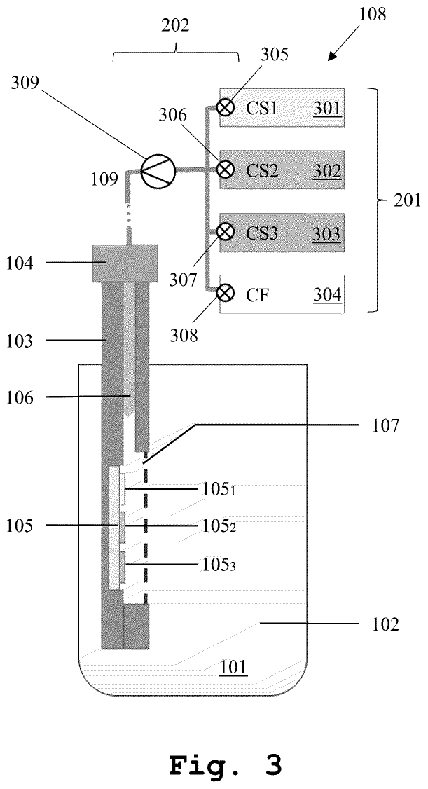

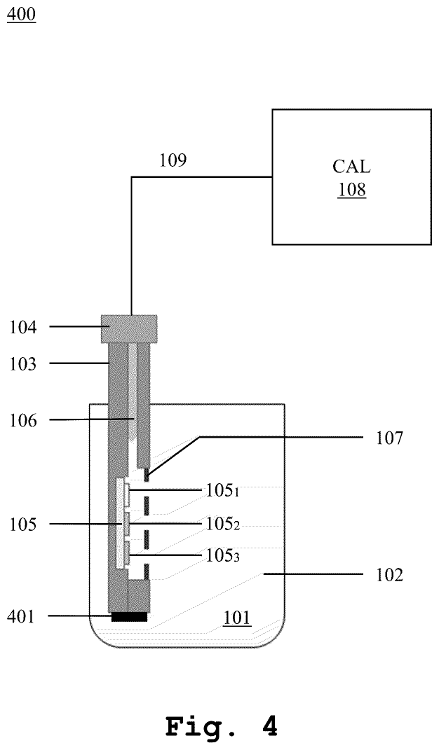

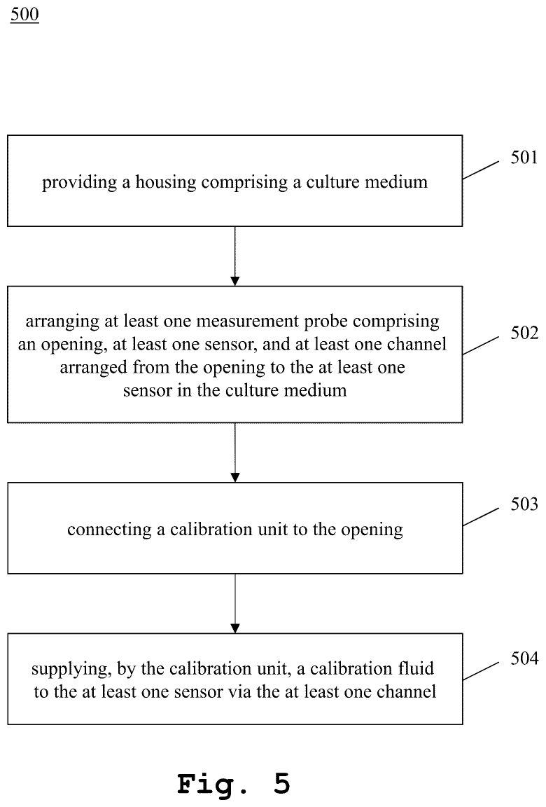

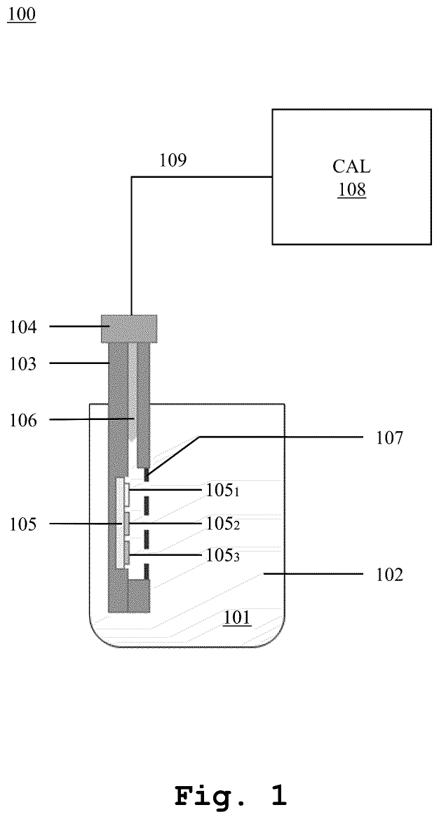

In FIG. 1, a first embodiment of the measurement system 100 according to the first aspect of the disclosure is illustrated. The measurement system 100 may comprise a housing 101, e.g., a bioreactor vessel, comprising a cell-culture medium 102 and a measurement probe 103 arranged in the cell-culture medium 102, for example inside of the housing 101.

The measurement probe 103 may comprise an opening 104, an array of sensors 105, herein exemplarily shown with three sensors 105₁, 105₂, 105₃, and a channel 106 arranged from the opening 104 to the array of sensors 105, especially within the measurement probe 103.

The measurement probe 103 may further comprise a membrane 107 encompassing the array of sensors 105, for example to protect the array of sensors 105 from the cell-culture medium 102. For instance, the membrane 107 may be arranged, i.e., the position of the membrane 107 may be aligned, with respect to the position/location of the array of sensors 105 in the measurement probe 103.

For example, the probe material, especially of the body of the measurement probe 103, may differ from the membrane material. In this regard, the measurement probe 103 may comprise a slot or an aperture, for example on the body of the measurement probe 103, corresponding to the position/location of the array of sensors 105, and the membrane 107 may be attached to the measurement probe 103, for example to the body of the measurement probe 103, at the slot or the aperture, for example covering the slot or the aperture.

The measurement system 100 may further comprise a calibration unit 108 connected to the opening 104 of the measurement probe 103 and further to the channel 106 inside of the measurement probe 103, for example via a flow path 109. The calibration unit 108 may supply a calibration fluid to the array of sensors 105 through the channel 106, e.g., via the flow path 109 through the opening 104 of the measurement probe 103.

In this regard, the membrane 107 may confine the calibration fluid above the array of sensors 105, for example during the calibration measurement, and may prevent rapid diffusion and dilution of the calibration fluid into the housing 101.

For example, the membrane 107 may comprise pores having an internal width sufficient to pass molecules, such as proteins, through the membrane 107. For instance, the pores may have an internal width of about 1 micrometer to 5 micrometers, for example an internal width of about 10 nm to 100 nm, to allow sample diffusion to the array of sensors 105 while preventing cells to contaminate the array of sensors 105, for example the surface of the sensors.

In other words, the membrane 107 may correspond to or be a one-way flow membrane, which may restrict the flow of the calibration fluid from the channel 106 and/or the array of sensors 105 of the measurement probe 103 into the housing 101. However, the membrane 107 may allow sample diffusion, such as protein molecules, to the array of sensors 105, for example by allowing the sample to flow from the housing 101, e.g., through the pores, into the measurement probe 103.

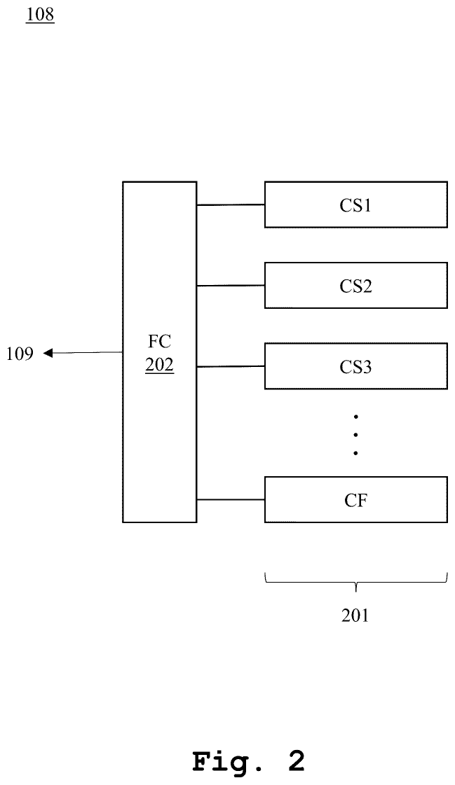

In FIG. 2, an embodiment of the calibration unit 108 is illustrated. The calibration unit 108 may comprise a storage section 201 and a flow controller 202 connected to the storage section 201. In this regard, the storage section 201 may act as a reservoir to store one or more calibration fluids, such as the calibration solution CS1, the calibration solution CS2, the calibration solution CS3, and cleaning fluids CF.

For example, the calibration solution CS1, the calibration solution CS2, and the calibration solution CS3 may respectively correspond to the sensor 105₁, the sensor 105₂, the sensor 105₃ of the array of sensors 105, e.g., for a respective calibration species. Alternatively, the calibration solution CS1, the calibration solution CS2, and the calibration solution ‘CS3 may comprise different concentrations of a mixture of calibration species.

The flow controller 202 may comprise flow controlling means, such as a set of valves and a pump, that may be actuated on demand, e.g., via a software interface. As such, the flow controller 202 may push the calibration solution CS1, the calibration solution CS2, the calibration solution CS3, and/or the cleaning fluid CF through the channel 106 to the array of sensors 105, e.g., via the flow path 109 through the opening 104 of the measurement probe 103.

Furthermore, the flow controller 202 may pull a sample from the housing 101 through the membrane 107 onto the array of sensors 105, for example to control the flow during the measurement.

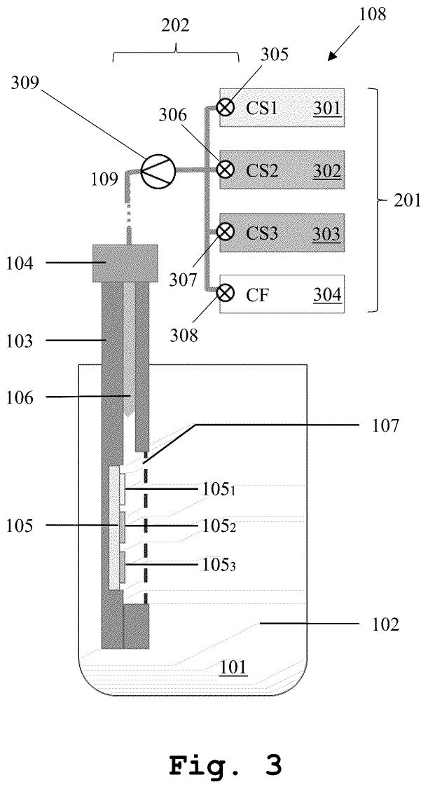

In FIG. 3, an in situ calibration operation of the measurement system 100 is illustrated. For example, the array of sensors 105 may comprise the sensor 105₁, the sensor 105₂, the sensor 105₃; each may measure or monitor a respective single physical or chemical or biochemical parameter.

The calibration unit 108, for example the storage section 201, may comprise a first calibration solution storage 301 containing a first calibration solution CS1 corresponding to the sensor 105₁, a second calibration solution storage 302 containing a second calibration solution CS2 corresponding to the sensor 105₂, a third calibration solution storage 303 containing a third calibration solution CS3 corresponding to the sensor 105₃, and a fourth storage 304 containing the cleaning fluid CF. The calibration solution CS1, the calibration solution CS2, the calibration solution CS3, and the cleaning fluid CF may be sterile.

The flow controller 202 may comprise a first valve 305, e.g., a directional valve, connected to the first calibration solution storage 301 to control a flow of the first calibration solution CS1, a second valve 306 connected to the second calibration solution storage 302 to control a flow of the second calibration solution CS2, a third valve 307 connected to the third calibration solution storage 303 to control a flow of the third calibration solution CS3, and a fourth valve 308 connected to the fourth storage 304 to control a flow of the cleaning fluid CF.

The flow controller 202 may further comprise a pump 309 connected to the first valve 305, the second valve 306, the third valve 307, and the fourth valve 308, and further to the flow path 109. In this regard, the pump 309 may push one or more of the calibration solution CS1, the calibration solution CS2, the calibration solution CS3, or the cleaning fluid CF to the sensor 105₁, the sensor 105₂, the sensor 105₃, via the channel 106 through the opening 104 of the measurement probe 103. The first valve 305, the second valve 306, the third valve 307, the fourth valve 308, and the pump 309 may be actuated on demand, e.g., via a software interface.

For instance, if a calibration measurement for the sensor 105₁ is needed, the first valve 305 may be operated or activated to enable a flow of the first calibration solution CS1, and the pump 309 may push the first calibration solution CS1 via the flow path 109 through the opening 104 and further through the channel 106 to the sensor 105₁.

Analogously, if the sensor 105₁ is required to be cleaned, the fourth valve 308 may be operated or activated to enable a flow of the cleaning fluid CF, and the pump 309 may push the cleaning fluid CF via the flow path 109 through the opening 104 and further through the channel 106 to the sensor 105₁.

For instance, in case of a parameter measurement, the pump 309 may pull a sample from the housing through the membrane 107 onto the sensor 105₁, the sensor 105₂, and the sensor 105₃. In this regard, the membrane 107 may comprise a uniform surface or may comprise different surfaces corresponding respectively to the sensor 105₁, the sensor 105₂, and the sensor 105₃, e.g., with different pore sizes, for example to allow selective measurements.

It is to be noted that the opening 104 of the measurement probe 103 may additionally act as a sealing around the flow path 109 connecting the channel 106 within the measurement probe 103. Since the measurement probe 103, the calibration solution CS1, the calibration solution CS2, the calibration solution CS3, and the cleaning fluid CF may be sterilized before use, the in situ calibration may not affect the sterility of the measurement probe 103 and/or the housing 101.

Moreover, since the first valve 305, the second valve 306, the third valve 307, the fourth valve 308, and the pump 309 may be actuated via a software interface, in situ automated calibration of the measurement probe 103, for example of the array of sensors 105, can be achieved without needing to collect samples.

For example, for calibrating the sensor 105₁, the sensor 105₂, the sensor 105₃, the calibration solutions CS1, the calibration solution CS2, and the calibration solution CS3 may be fed to the sensor 105₁, the sensor 105₂, and the sensor 105₃, which correspond to a desired or preset value or data of the sensor 105₁, the sensor 105₂, and the sensor 105₃. Using the calibration solution CS1, the calibration solution CS2, and the calibration solution CS3, one or more measurement values or data may be collected from the sensor 105₁, the sensor 105₂, and the sensor’ 105₃, and may be compared with the desired or preset value or data. Afterwards, an adjustment or a set of adjustments may be performed to ensure the measurement accuracy of the sensor 105₁, the sensor 105₂, and the sensor’ 105₃.

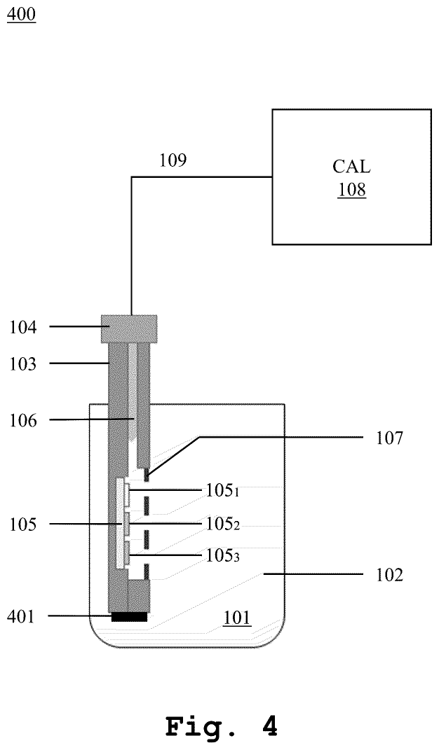

In FIG. 4, a second embodiment of the measurement system 400 according to the first aspect of the disclosure is illustrated. The measurement system 400 differs from the measurement system 100 in that the measurement probe 103 of the measurement system 400 may additionally comprise a further sensor or an additional array of sensors 401, for example arranged at the measurement probe 103 to be in direct contact with the cell-culture medium 102.

For example, the further sensor or the additional array of sensors 401 may be arranged at the measurement probe 103 outside of the membrane 107. In other words, the membrane 107 might not encompass the further sensor or the additional array of sensors 401, for example to facilitate differential measurements.

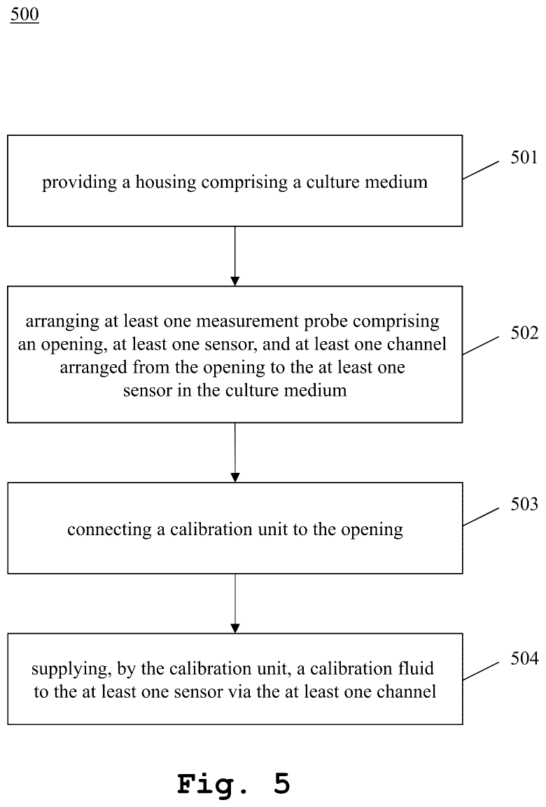

In FIG. 5, an embodiment of the method 500 according to the second aspect of the disclosure is illustrated. In a first step 501, a housing comprising a culture medium is provided. In a second step 502, at least one measurement probe comprising an opening, at least one sensor, and at least one channel arranged from the opening to the at least one sensor is arranged in the culture medium. In a third step 503, a calibration unit is connected to the opening of the at least one measurement probe. In a fourth step 504, a calibration fluid is supplied by the calibration unit to the at least one sensor via the at least one channel.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A measurement system comprising:
a housing comprising a culture medium and a measurement probe arranged in the culture medium, wherein the measurement probe comprises an opening, a sensor, and a channel arranged from the opening to the sensor; and
a calibration unit connected to the opening and configured to supply a calibration fluid to the sensor via the channel.

2. The measurement system according to claim 1, wherein the measurement probe comprises a membrane encompassing the sensor and configured to confine the calibration fluid at or around the sensor.

3. The measurement system according to claim 2, wherein the membrane comprises a plurality of pores with an internal width from 1 micrometer to 5 micrometers.

4. The measurement system according to claim 2, wherein the membrane comprises a plurality of pores with an internal width from 10 nm to 100 nm.

5. The measurement system according to claim 2, wherein the calibration unit comprises a controller configured to control a flow of the calibration fluid in the channel.

6. The measurement system according to claim 5, wherein the controller is further configured to control a second flow of the culture medium to the sensor through the membrane.

7. The measurement system according to claim 1, wherein the calibration fluid comprises a calibration solution with a defined concentration.

8. The measurement system according to claim 1, wherein the calibration fluid comprises a plurality of calibration solutions with different concentrations.

9. The measurement system according to claim 1, wherein the calibration unit is further configured to supply a cleaning fluid to the sensor via the channel.

10. The measurement system according to claim 9, wherein the measurement probe is sterile.

11. The measurement system according to claim 9, wherein the calibration fluid is sterile.

12. The measurement system according to claim 9, wherein, and the cleaning fluid is sterile.

13. The measurement system according to claim 1, wherein the sensor is a first sensor and the measurement probe further comprises a second sensor configured to be in contact with the culture medium.

14. The measurement system according to claim 13,
wherein the first sensor and/or the second sensor are electrochemical sensors.

15. The measurement system according to claim 14, wherein the first sensor and/or the second sensor are configured to measure a physical parameter, a chemical parameter, and a biochemical parameter of the culture medium.

16. The measurement system according to claim 1, wherein the housing is a cell culture vessel.

17. A method comprising:
providing a housing comprising a culture medium;
arranging a measurement probe comprising an opening, a sensor, and a channel arranged from the opening to the sensor in the culture medium;
connecting a calibration unit to the opening; and
supplying, by the calibration unit, a calibration fluid to the sensor via the channel.

18. The method according to claim 17, further comprising providing a membrane encompassing the sensor to confine the calibration fluid at or around the sensor.

19. The method according to claim 17, wherein the calibration unit comprises a controller, the method further comprising:
controlling, via the controller, a flow of the calibration fluid in the channel.

20. The method of claim 19, further comprising controlling, via the controller, a flow of the culture medium to the sensor through the membrane.
